(19) United States
(12) Patent Application Publication
(10) Pub. No.: US 2026/0235614 A1
(43) Pub. Date: Aug. 13, 2026

(54) METHODS OF IDENTIFYING AND EVALUATING GENES FOR INSECT CONTROL

(71) Applicant: BASF Agricultural Solutions US LLC, Research Triangle Park, NC (US)

(72) Inventors: Mahmoud Kamal Ahmadi, Medford, MA (US); Jason Anthony Goebel, Cary, NC (US); Sara Lis Milam Lenzen, Cary, NC (US); Alberto Bressan, Durham, NC (US); Jessica Monserrate, Durham, NC (US)

(73) Assignee: BASF Agricultural Solutions US LLC, Research Triangle Park, NC (US)

(21) Appl. No.: 19/241,550

(22) PCT Filed: Dec. 18, 2023
(86) PCT No.: PCT/US2023/084515

§ 371 Date: Jun. 18, 2025

Related U.S. Application Data
(60) Provisional application No. 63/433,851, filed on Dec. 20, 2022.

(51) Int. Cl. G01N 33/68

(52) U.S. Cl. CPC G01N 33/68; G01N 2333/43526

(57) ABSTRACT

Methods and compositions for identifying pesticidal genes are provided. The methods of the embodiments comprise systematically designing brush border membrane vesicles (BBMV) from specific target pest wherein the BBMV is filled with a detectable substance and then bringing in contact with said BBMV a candidate pesticidal protein.

### CROSS-REFERENCE TO RELATED APPLICATIONS

This application claims priority to U.S. Provisional Application No. 63/433,851 filed on Dec. 20, 2022, the entire contents of which are hereby incorporated by reference.

### BACKGROUND

Insect pests are a major factor in the loss of the world's agricultural crops. For example, corn rootworm feeding damage and boll weevil damage can be economically devastating to agricultural producers. Insect pest-related crop loss from corn rootworm alone has reached one billion dollars a year.

Certain species of microorganisms of the genus Bacillus are known to possess pesticidal activity against a broad range of insect pests including Lepidoptera, Diptera, Coleoptera, Hemiptera, and others. Bacillus thuringiensis and Bacillus popilliae are among the most successful biocontrol agents discovered to date. Insect pathogenicity has been attributed to strains of: B. larvae, B. lentimorbus, B. popilliae, B. sphaericus, B. thuringiensis (Harwook, ed. (1989) Bacillus (Plenum Press), p. 306) and B. cereus (International Publication No. WO 96/10083). Pesticidal activity appears to be concentrated in parasporal crystalline protein inclusions, although pesticidal proteins have also been isolated from the vegetative growth stage of Bacillus. Several genes encoding these pesticidal proteins have been isolated and characterized (see, for example, U.S. Pat. Nos. 5,366,892 and 5,840,868).

Microbial pesticides, particularly those obtained from Bacillus strains, have played an important role in agriculture as alternatives to chemical pest control. Pesticidal proteins isolated from strains of Bacillus thuringiensis, known as 8-endotoxins or Cry toxins, are initially produced in an inactive protoxin form. These protoxins are lytically converted into an active toxin through the action of proteases in the insect gut. See, Rukmini et al. (2000) Biochimie 82:109-116; Oppert (1999) Arch. Insect Biochem. Phys. 42:1-12; and Carroll et al. (1997) J. Invertebrate Pathology 70:41-49. Proteolytic activation of the toxin can include the removal of the N- and C-terminal peptides from the protein, as well as internal cleavage of the protein. Once activated, the Cry toxin binds with high affinity to receptors on epithelial cells in the insect gut, thereby creating leakage channels in the cell membrane, lysis of the insect gut, and subsequent insect death through starvation and septicemia. See, e.g., Li et al. (1991) Nature 353:815-821.

Genetically engineering crop plants with pesticidal genes to produce pesticidal proteins from Bacillus (and other sources) is a key solution to a growing problem of feeding an ever-expanding world population. Insect pressure, expanding pest territory and insect resistance to control are all critical obstacles in securing the world's future food supply. With this scientist have put immense effort into the discovery of new genes and modes of action to keep ahead of these obstacles. It seems that nature provides an immense collection of tools that can be utilized for such purpose however discovery of such genes are costly and require a sorting of candidates to identify the safe and best performing genes that can be utilized in agriculture. Therefore, new methods for efficiently identifying novel pesticidal genes and quickly predicting their performance in planta are needed in the art.

### BRIEF DESCRIPTION OF THE DRAWINGS

FIG. 1A is a bar graph showing the results of a FAW gut leakage assay with ARP166 variants.

FIG. 1B is a bar graph showing the results of a FAW whole body leakage assay with ARP166 variants.

FIG. 2 is a bar graph showing the results of an Hz gut leakage assay with ARP540 variants.

FIG. 3 is a bar graph showing the results of an Hz gut leakage assay with ARP793 variants.

### SUMMARY

The present embodiments provide for methods and compositions for identifying novel pesticidal genes and in predicting the performance of such pesticidal genes in a plant against a respective plant pest. The methods disclosed herein permit the rapid and efficient screening of a large number of pesticidal genes (and their gene products) to identify potential pesticidal genes to be utilized in transgenic crops for the control of insects. The methods for identifying novel pesticidal gene comprise systematically designing and constructing a brush border membrane vesicle (herein, “BBMV”) wherein the brush border member used to make the BBMV is derived from a specific plant pest of interest (e.g. Fall armyworm, corn earworm, etc.). Inserted within the lumen of this BBMV is inserted either a dye, a reporter gene or any means that can be visually or detected in solution if the BBMV outer membrane begins to leak or is punctured (herein “detectable(s)”). This BBMV can be used to quickly screen against potential pesticidal gene candidates in a high-throughput manner where various gene candidates are applied to the BBMV and if leakage is detected it can be deduced that the candidate gene is active against the respective insect for which the brush border membrane was derived. This method eliminates the need to run arduous amounts of insect bioassays or do in planta studies. The degree of leakage can also be correlated with degree of effectiveness of a gene candidate against a give crop pest. The methods provided herein, help to quickly eliminate ineffective pesticidal genes against a given pest from a pool of candidate genes in a pesticidal gene discovery program. Pesticidal genes causing leakage of BBMV can be identified and further studies conducted to further characterize as well as transform into respective crop plants relative to the targeted pest. The methods of the embodiments are further amenable to automation and high throughput screening.

### DETAILED DESCRIPTION

One or more embodiments are drawn to compositions and methods for identifying and predicting performance of novel pesticidal proteins that show resistance against a given pest. By “resistance” is intended that the pest (e.g., insect) is killed upon ingestion or other contact with the polypeptides of one or more embodiments. By “tolerance,” it is intended to mean an impairment or reduction in the movement, feeding, reproduction, or other functions of the pest. The methods comprise the use of BBMV with a detectable inserted in the lumen of the BBMV. The BBMV comprises the brush border membrane of a pest of interest (herein, “target pest”). In one embodiment, the target pest is comprised from the orders Lepidoptera, Diptera, Hemiptera, and Coleoptera and Nematoda. This BBMV is then put in contact with a candidate pesticidal gene (herein, “candidate gene”). If the candidate gene is active against the BBMV, it will attach to the BBMV and cause the lysis of the outer membrane of the BBMV. This lysis will then allow detectable to leak out of the BBMV into solution which then can be detected and analyzed. This method allows for the rapid evaluation of gene candidates activity and effectiveness against a target pest.

In another embodiment, a specific receptor(s) can be embedded into the membrane of the BBMV in an insect cell line (for example Sf9), one could isolate the cell membrane, and then prepare the BBMV form these cell membranes. This new approach is referred to as the Receptor Specific Proteoliposome (RS-PT) assay. Applicants have tested this assay for two pair of receptors pesticidal proteins (ABCC2-Cry1 Ac and ABCB1-Axmi22z). The results are very promising indicating that one skilled in the art could quickly evaluate varying collections of known or novel receptors from insect pest gut lining that could serve as targets for pesticidal proteins. The RS-PT approach allows one skilled in the art, for example, to also identify new novel modes of action by way of applying pesticidal proteins to BBMVs having specific insect gut receptors embedded and carry out binding affinity studies to identify new or novel genes that might react with such receptor to form a pore in the BBMV. In one embodiment, the receptor can be a protein receptor is comprised from gustatory receptors, odorant receptors, ionotropic receptors, transient receptors, potential channel receptors, neuropeptide receptors and serotonin receptors.

By “pesticidal toxin” or “pesticidal protein,” it is intended to mean a toxin that has toxic activity against one or more pests, including, but not limited to, members of the Lepidoptera, Diptera, Hemiptera, and Coleoptera orders, or the Nematoda phylum, or a protein that has homology to such a protein. Pesticidal proteins include amino acid sequences deduced from the full-length nucleotide sequences disclosed herein, and amino acid sequences that are shorter than the full-length sequences, either due to the use of an alternate downstream start site, or due to processing that produces a shorter protein having pesticidal activity. Processing may occur in the organism the protein is expressed in, or in the pest after ingestion of the protein.

In another aspect, the BBMV concept as described herein can be used to evaluate chemicals or other active ingredients against target pest. For example, one could provide a screen of novel chemicals and test against a collection of BBMVs in a multi-well plates and test for BBMV leakage which would indicate activity or effectiveness of chemicals on the brush border membrane of a target pest.

In another aspect, one skilled in the art could use methods described herein to evaluate and characterize pesticidal proteins. For example, one could create fragments of a known or novel pesticidal protein to evaluate what portions of said pesticidal protein binds and cleaves the brush border membrane of a target pest.

In another aspect, one skilled in the art could quickly evaluate the effectiveness of pesticidal protein variants on a given target pest. For example, one skilled in the art might create multiple variants of a given pesticidal gene with the intention to expand the breadth of target pest the said pesticidal gene can control and/or the effectiveness of said pesticidal gene. In this example the methods described herein can be used to quickly screen such variants against BBMVs derived from one or more target pest. Variants include polypeptides that differ in amino acid sequence due to mutagenesis.

In one aspect, one or more embodiments are antibodies which may be used to quantitatively or qualitatively detect a protein or peptide molecules of interest, or to detect post translational modifications of the proteins. As used herein, an antibody or peptide is said to “specifically bind” to a protein or peptide molecule of the embodiments if such binding is not competitively inhibited by the presence of non-related molecules. In some aspects, the BBMV's of the embodiments can be used to detect a pesticidal protein of interest through the use of antibodies. Methods for utilizing antibodies for the detection of a protein or peptide of interest are known in the art.

In another aspect, BBMVs of the embodiments can be used to evaluate effectiveness of a given expression cassette. For example, a given pesticidal gene with known activity against a target pest could be used to evaluate the effectiveness of various post translation strategies thus indicating optimal ways to express and/or target said pesticidal genes in plants to control a target pest. Another example, one skilled in the art might evaluate the effectiveness of gene promoters utilizing the methods described herein including inducible promoters where a pesticidal gene is operably linked to a candidate inducible promoter and various screens could be carried out by adding various inducing agents. If the BBMV forms a leak one could then correlate an inducible promoter with its respective inducing agents which could then be employed in transgenic plant expression of genes.

By “plant,” it is intended to mean whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic plants” or “transformed plants” or “stably transformed” plants or cells or tissues refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell. “Heterologous” generally refers to the nucleic acid sequences that are not endogenous to the cell or part of the native genome in which they are present, and have been added to the cell by infection, transfection, microinjection, electroporation, microprojection, or the like.

Among such DNA sequences encoding proteins having various pesticidal resistance of tolerance to insects include the Cry1F protein or hybrids derived from a Cry1F protein (e.g., the hybrid Cry1A-Cry1F proteins described in U.S. Pat. Nos. 6,326,169; 6,281,016; 6,218,188, or toxic fragments thereof), the Cry1A-type proteins or toxic fragments thereof, the Cry1 Ac protein or hybrids derived from the Cry1Ac protein (e.g., the hybrid Cry1Ab-Cry1Ac protein described in U.S. Pat. No. 5,880,275) or the Cry1 Ab or Bt2 protein or insecticidal fragments thereof as described in EP451878, the Cry2Ae, Cry2Af or Cry2Ag proteins as described in WO2002/057664 or toxic fragments thereof, the Cry1A.105 protein described in WO 2007/140256 (SEQ ID No. 7) or a toxic fragment thereof, the VIP3Aa19 protein of NCBI accession ABG20428, the VIP3Aa20 protein of NCBI accession ABG20429 (SEQ ID No. 2 in WO 2007/142840), the VIP3A proteins produced in the COT202 or COT203 cotton events (WO2005/054479 and WO2005/054480, respectively), the Cry proteins as described in WO2001/47952, the VIP3Aa protein or a toxic fragment thereof as described in Estruch et al. (1996), Proc. Natl. Acad. Sci. USA 28: 93 (11): 5389-94 and U.S. Pat. No. 6,291,156, the insecticidal proteins from Xenorhabdus (as described in WO98/50427), Serratia (particularly from S. entomophila) or Photorhabdus species strains, such as Tc-proteins from Photorhabdus as described in WO98/08932 (e.g., Waterfield et al., 2001, Appl. Environ. Microbiol. 67 (11): 5017-24; French-Constant and Bowen, 2000, Cell Mol Life Sci.; 57 (5): 828-33). Any variants or mutants of any one of these proteins differing in some (1-10, or 1-5) amino acids from any of the above could be effective against target, or which are fused to a transit peptide, such as a plastid transit peptide, or another protein or peptide, is included herein.

In various embodiments, the methods described herein could be used to identify ideal combinations of pesticidal proteins against a target pest or target pests. The methods could also be used to measure any negative impacts on pesticidal gene performance in combination with other genes or agents potentially indicating unfavorable combinations that might impact the effectiveness of the pesticidal gene. For example, such combinations could include genes for traits, such as herbicide tolerance, insect tolerance, drought tolerance, nematode control, water use efficiency, nitrogen use efficiency, improved nutritional value, disease resistance, improved photosynthesis, improved fiber quality, stress tolerance, improved reproduction, and the like or in combination with certain chemicals, hormones, or other agents. It is contemplated that the impact of environmental variables such as heat, alkalinity/acidity could be evaluated for their impact on pesticidal gene(s) and their respective performance.

Typically, this “plant expression cassette” will be inserted into a “plant transformation vector.” This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors.” Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the pesticidal gene are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

Active ingredients that might be used or evaluated in the present embodiments are normally applied in the form of compositions and can be applied to solution that comes in contact with BBMVs of the embodiments, simultaneously or in succession, with other compounds. These compounds can be fertilizers, weed killers, cryoprotectants, surfactants, detergents, pesticidal soaps, dormant oils, polymers, and/or time-release or biodegradable carrier formulations that permit long-term dosing of a target area following a single application of the formulation. They can also be selective herbicides, chemical insecticides, virucides, microbicides, amoebicides, pesticides, fungicides, bacteriocides, nematocides, molluscicides or mixtures of several of these preparations, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise, the formulations may be prepared into edible “baits” or fashioned into pest “traps” to permit feeding or ingestion by a target pest of the pesticidal formulation.

“Pest” includes but is not limited to, insects, fungi, bacteria, nematodes, mites, ticks, and the like. Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., and also Coleoptera, Lepidoptera, and Diptera.

The order Coleoptera includes the suborders Adephaga and Polyphaga. Suborder Adephaga includes the superfamilies Caraboidea and Gyrinoidea, while suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simuliidae, Bibionidae, and Cecidomyiidae. Suborder Brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and Calyptratae. Section Acalyptratae includes the families Otitidae, Tephritidae, Agromyzidae, and Drosophilidae. Section Calyptratae includes the families Hippoboscidae, Oestridae, Tachinidae, Anthomyiidae, Muscidae, Calliphoridae, and Sarcophagidae.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Nematodes include parasitic nematodes such as root-knot, cyst, and lesion nematodes, including Heterodera spp., Meloidogyne spp., and Globodera spp.; also including members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pallida (potato cyst nematodes). Lesion nematodes include Pratylenchus spp.

Hemipteran pests (which include species that are designated as Hemiptera, Homoptera, or Heteroptera) include, but are not limited to, Lygus spp., such as Western tarnished plant bug (Lygus hesperus), the tarnished plant bug (Lygus lineolaris), and green plant bug (Lygus elisus); aphids, such as the green peach aphid (Myzus persicae), cotton aphid (Aphis gossypii), cherry aphid or black cherry aphid (Myzus cerasi), soybean aphid (Aphis glycines Matsumura); brown plant hopper (Nilaparvata lugens), and rice green leafhopper (Nephotettix spp.); and stink bugs, such as green stink bug (Acrosternum hilare), brown marmorated stink bug (Halyomorpha halys), southern green stink bug (Nezara viridula), rice stink bug (Oebalus pugnax), forest bug (Pentatoma rufipes), European stink bug (Rhaphigaster nebulosa), and the shield bug Troilus luridus.

Insect pests of the embodiments for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscurus, grass thrips; Solenopsis molesta, thief ant; Tetranychus urticae, twospotted spider mite; Sorghum: Chilo partellus, sorghum borer; Spodoptera frugiperda, fall armyworm; Spodoptera cosmioides; Spodoptera eridania; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cepheus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Frankliniella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Spodoptera cosmioides; Spodoptera eridania; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Chilo suppressalis, Asiatic rice borer; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Spodoptera cosmioides; Spodoptera eridania; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Euschistus heros, neotropical brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

The following examples are offered by way of illustration and not by way of limitation.

### Examples

### Materials and Methods: BBMV (Brush Border Membrane Vesicles)

BBMV material was prepared from dissected 3rd or 4th instar Spodoptera frugiperda, fall armyworm (herein, “FAW”), Helicoverpa zea, corn earworm (herein, “Hz”), or Heliothis virescens, tobacco budworm (herein, Hv) as described by Wolfersberger with adjustments (1987).

FAW BBMV insect material was also prepared from entire insect (whole body, WB). The same Wolfersberger protocol was followed until after the first MgCl₂ precipitation step. The pellets were resuspended in half strength MET with 12 mM MgCl₂. BBMVs were loaded onto a 30/40/45% sucrose gradient containing 12 mM MgCl₂. The gradients were centrifuged at 27,000 rpm for 1 hour at 4 C. The band above 45% sucrose was removed and diluted 1:10 with half strength MET and 12 mM MgCl₂. The material was centrifuged again at 30,000×g for 30 min at 4° C. The supernatant was removed, each pellet was resuspended in half strength MET, homogenized, aliquoted, and flash frozen with liquid nitrogen. A BCA Assay was used to determine the concentration of the vesicles.

### Protein Sample Preparation

ARP166 and its variants were transformed into BL21 Gold cells. Cultures were grown in LB at 37° C. until OD600 nm was about 0.6-0.8. Protein expression was induced with 1 mM IPTG and the temperature was reduced to 18 C overnight. Cells were harvested and frozen until needed. Cells were lysed with BugBuster and soluble fusion protein was isolated with an MBP affinity column. The buffer was 50 mM Hepes pH 8, 200 mM NaCl, 10 mM maltose. Protein concentration was determined using gel densitometry.

ARP540 and its variants were transformed into T7 cells. Cultures were grown in instant TB in a 48-well block for 24 hour at 37° C. To pelleted cells, 100 μl of 0.1 mm glass beads and 250 μl of 50 mM Hepes pH 8, 200 mM NaCl, 5 mM EDTA were added. Samples were bead beaten for 3 min ×2 with a 1 minute rest in between. Cell debris was pelleted at 4000 rpm for 10 min. The soluble material was used in the bbmv leakage assay. Protein expression was checked via SDS PAGE.

ARP793 and its variants were transformed into BL21 Star. Cultures were grown in LB at 37 C until OD600 nm was ~0.6-0.7. Protein expression was induced with 0.1 mM IPTG and the temperature was reduced to 18 C overnight. Cell were harvested and frozen until needed. Cells were resuspended in 50 mM sodium carbonate pH 10.5 and lysed with micro fluidics apparatus. Soluble fusion protein was isolated with an MBP affinity column. The buffer was 50 mM sodium carbonate pH 10.5, 10 mM maltose. The protein concentration was determined by BCA Assay and gel densitometry.

### FAW Leakage Assay

The appropriate amount of dye solution (80 mM 5 (6)-carboxyfluorescein, 1 μM CHAPS, 100 mM Hepes pH 7.3, 200 mM NaCl, 5 mM EDTA, 0.26 N NaOH) was added to thawed, pelleted FAW bbmv. The dye was encapsulated by alternating vortex and sonication 3 times for 30 sec. The encapsulated vs free dye was separated by SEC (PD10 column, Superdex 75 10/300 GL, or HiLoad Superdex 75 pg 16/600). The amount of BBMV was determined via BCA Assay. The positive fractions were pooled and diluted to 0.05 mg/ml BBMV. Trypsin, if necessary, was added to the encapsulated bbmv at 0.1 mg/ml. Protein and encapsulate BBMV were mixed and the fluorescence signal (excitation 480 nm, emission 520 nm) was monitored for 3 hours. Once the run was completed, 1% triton was added to achieve the total leakage signal.

### Hz Leakage Assay

The appropriate amount of dye solution (80 mM calcein, 1 μM CHAPS, 50 mM Hepes pH 8, 200 mM NaCl, 5 mM EDTA, 0.33 N NaOH) was added to thawed, pelleted Hz bbmv. The dye was encapsulated by alternating vortex and ice 3 times for 30 sec. The encapsulated vs free dye was separated by SEC (G50 column, PD10 column, Superdex 75 10/300 GL, or HiLoad Superdex 75 pg 16/600). The amount of bbmv was determined via BCA Assay. The positive fractions were pooled and diluted to 0.05 mg/ml bbmv. The bbmvs were equilibrated on ice for at least 1 hour. If necessary, trypsin was added to increase the speed of the reaction (0.1 mg/ml for ARP540 or 0.02 mg/ml for ARP793). Protein and bbmv were mixed and the fluorescence signal (excitation 480 nm, emission 520 nm) was monitored for 5 hours. Once the run was completed, 1% triton was added to achieve the total leakage signal.

### Hv Leakage Assay

The appropriate amount of dye solution (80 mM calcein, 1 μM CHAPS, 50 mM Hepes pH 8, 250 mM trehalose, 1×HALT, 1 mM PMSF, 0.33 N NaOH) was added to thawed, pelleted Hv bbmv. The dye was encapsulated by alternating vortex and ice 3 times for 30 sec. The encapsulated vs free dye was separated by SEC (G50 column). The amount of bbmv was determined via BCA Assay. The positive fractions were pooled and diluted to 0.05 mg/ml. The bbmvs were equilibrated on ice for at least 30 min. Protein and bbmv were mixed and the fluorescence signal (excitation 480 nm, emission 520 nm) was monitored for 1 hour.

### Results

FAW Leakage Assay with ARP166 Improved Variant Detection

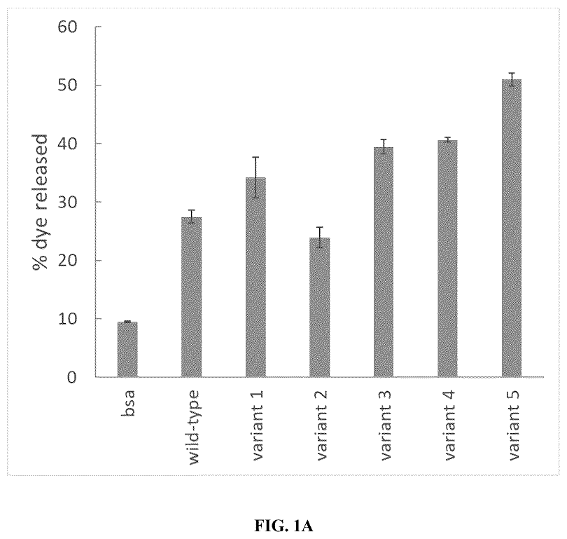

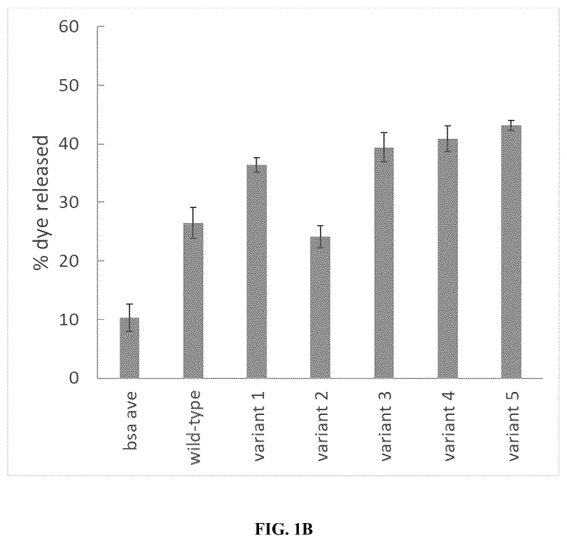

Purified fusion ARP166 and five variants were tested in the FAW gut and WB leakage assay, as well as FAW insect bioassay. The EC50 was collected for each variant and the wild-type protein (Table 1A). The variants were then ranked/grouped based on the magnitude of improvement from the wild-type protein. These same variants were tested in both the FAW gut (Table 1B and FIG. 1A) and WB (Table 1C and FIG. 1B) leakage assay. The graph shows the percent release of dye from the bbmv at time 30 min. The error bars are standard deviation from 3 biological replicates. These values are also shown in the table with ranking assigned based on the change of improvement from wild-type protein. Table 1D shows a comparison of all 3 FAW assays.

TABLE 1A
FAW EC50 data for wild-type and variants.

| | EC50 | Fold | |
|---|---|---|---|
| ARP166 | (μg / ml) | Change | Ranking |
| Wild-type | 1698 | — | — |
| Variant 1 | 152 | 11 | 3 |
| Variant 2 | 1395 | 1 | — |
| Variant 3 | 94 | 18 | 2 |
| Variant 4 | 21 | 81 | 1 |
| Variant 5 | 77 | 22 | 2 |

TABLE 1B
FAW gut leakage assay data at time
30 min for wild-type and variants

| | FAW Gut Dye | % | |
|---|---|---|---|
| ARP166 | Released | Change | Ranking |
| Wild-type | 27 | — | — |
| Variant 1 | 34 | 26 | 3 |
| Variant 2 | 24 | −11 | — |
| Variant 3 | 40 | 48 | 2 |
| Variant 4 | 41 | 52 | 2 |
| Variant 5 | 51 | 89 | 1 |

TABLE 1C
FAW WB leakage assay data at time
30 min for wild-type and variants

| | FAW WB Dye | % | |
|---|---|---|---|
| ARP166 | Released | Change | Ranking |
| Wild-type | 26 | — | — |
| Variant 1 | 36 | 38 | 3 |
| Variant 2 | 24 | −8 | — |
| Variant 3 | 39 | 50 | 2 |
| Variant 4 | 41 | 58 | 1 |
| Variant 5 | 43 | 65 | 1 |

TABLE 1D
Rankings for each detection method for wild-type and variants

| | EC50 | FAW Gut | FAW WB |
|---|---|---|---|
| ARP166 | Ranking | Ranking | Ranking |
| Wild-type | — | — | — |
| Variant 1 | 3 | 3 | 3 |
| Variant 2 | — | — | — |
| Variant 3 | 2 | 2 | 2 |
| Variant 4 | 1 | 2 | 1 |
| Variant 5 | 2 | 1 | 1 |

The rankings from each detection method are very similar, showing the power of the bbmv leakage assay. It is also important to note that the WB bbmv prep method yields a similar result to the gut bbmv prep method. This result does not happen without the additional processing of the WB bbmv with the sucrose gradient.

The insecticidal activity of variants was done via eye scoring and by comparing with untreated insects for size in a bioassay plate. The stunts were scored as either 0, 1, 2 3 or 4. A 0 score indicating no stunting, a score of 1 indicating 1-25% stunting, a score of 2 indicating 26-50% stunting, a score of 3 indicating 51-75% stunting and a score of 4 indicating 76-100% stunting.

Hz Leakage Assay with ARP540 Improved Variants

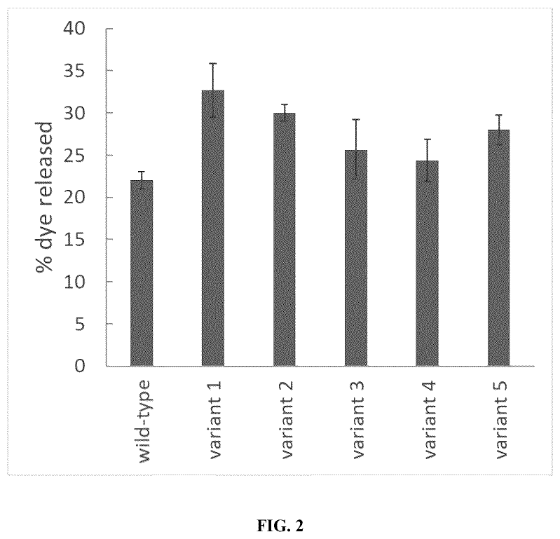

ARP540 and five variant lysates were tested in the Hz leakage assay, as well as the Hz insect bioassay. The EC50 was collected for each variant and wild-type protein (Table 2A). The variants were ranked/grouped based on the magnitude of improvement from the wild-type protein. These same variants were tested in the Hz leakage assay (Table 2B and FIG. 2). The graph shows the percent release of dye from the bbmv at time 3 hour. The error bars are standard deviation from 3 biological replicates. These values are also shown in the table with ranking assigned based on the change of improvement from wild-type protein. Table 2C shows a comparison of Hz insect bioassay to Hz leakage assay.

Again, the rankings are similar between the insect bioassay and the bbmv leakage assay, suggesting the leakage assay can be used to filter out improved variants.

The insecticidal activity of variants was done via eye scoring and by comparing with untreated insects for size in a bioassay plate. The stunts were scored as either 0, 1, 2 3 or 4. A 0 score indicating no stunting, a score of 1 indicating 1-25% stunting, a score of 2 indicating 26-50% stunting, a score of 3 indicating 51-75% stunting and a score of 4 indicating 76-100% stunting.

TABLE 2A
Hz EC50 data for wild-type and variants

| | EC50 | Fold | |
|---|---|---|---|
| ARP540 | (mg / ml) | Change | Ranking |
| Wild-type | 0.4 | — | — |
| Variant 1 | 0.001 | 400 | 1 |
| Variant 2 | 0.002 | 200 | 2 |
| Variant 3 | 0.02 | 40 | 3 |
| Variant 4 | 0.02 | 40 | 3 |
| Variant 5 | 0.001 | 400 | 1 |

TABLE 2B
Hz gut leakage data at time 3 hour for wild-type and variants

| | Hz Gut Dye | % | |
|---|---|---|---|
| ARP540 | Released | Change | Ranking |
| Wild-type | 22 | — | — |
| Variant 1 | 33 | 48 | 1 |
| Variant 2 | 30 | 36 | 2 |
| Variant 3 | 26 | 17 | 3 |
| Variant 4 | 24 | 11 | 3 |
| Variant 5 | 38 | 27 | 2 |

TABLE 2C
Rankings for each detection method for wild-type and variants

| | EC50 | Hz gut |
|---|---|---|
| ARP540 | ranking | ranking |
| Wild-type | — | — |
| Variant 1 | 1 | 1 |
| Variant 2 | 2 | 2 |
| Variant 3 | 3 | 3 |
| Variant 4 | 3 | 3 |
| Variant 5 | 1 | 2 |

Hz Leakage Assay with ARP793 Improved Variants

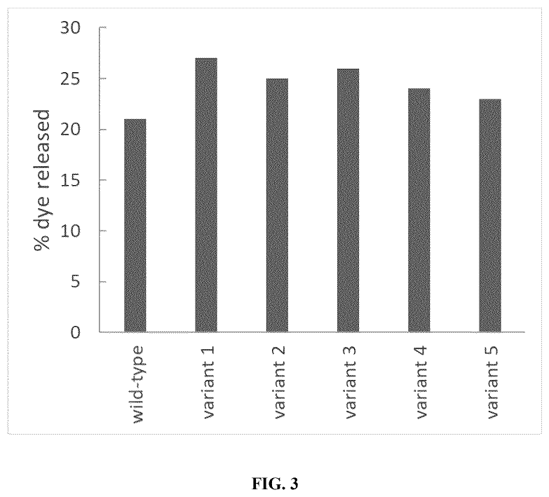

Purified fusion ARP793 and 5 variants were tested in the Hz leakage assay, as well as the Hz insect bioassay. The EC50 was collected for each variant and wild-type protein (Table 3A). The variants were ranked/grouped based on the magnitude of improvement from the wild-type protein. These same variants were tested in the Hz leakage assay (Table 3B and FIG. 3). The graph shows the percent release of dye from the bbmv at time 4 hour. These values are also shown in the table with ranking assigned based on the percent change of improvement from wild-type protein. Table 3C shows a comparison of the Hz insect bioassay to the Hz leakage assay.

Again, the rankings are similar between the insect bioassay and the bbmv leakage assay, suggesting the leakage assay can be used to filter out improved variants.

The insecticidal activity of variants was done via eye scoring and by comparing with untreated insects for size in a bioassay plate. The stunts were scored as either 0, 1, 2 3 or 4. A 0 score indicating no stunting, a score of 1 indicating 1-25% stunting, a score of 2 indicating 26-50% stunting, a score of 3 indicating 51-75% stunting and a score of 4 indicating 76-100% stunting.

TABLE 3A
Hz EC50 data for wild-type and variants

| | | | |
|---|---|---|---|
| | EC50 | Fold | |
| ARP793 | (mg / ml) | Change | Ranking |

| | | |
|---|---|---|
| Wild-type | — | — |
| Variant 1 | 23 | 1 |
| Variant 2 | 8 | 2 |
| Variant 3 | 9 | 2 |
| Variant 4 | 13 | 2 |
| Variant 5 | 3 | — |

TABLE 3B
Hz gut leakage data at time 3 hour for wild-type and variants

| | Hz Gut Dye | % | |
|---|---|---|---|
| ARP793 | Released | Change | Ranking |
| Wild-type | 21 | — | — |
| Variant 1 | 27 | 28 | 1 |
| Variant 2 | 25 | 19 | 2 |
| Variant 3 | 26 | 24 | 1 |
| Variant 4 | 24 | 14 | 2 |
| Variant 5 | 23 | 10 | 3 |

TABLE 3C
Rankings for each detection method for wild-type and variants

| | EC50 | Hz Gut |
|---|---|---|
| ARP793 | Ranking | Ranking |
| Wild-type | — | — |
| Variant 1 | 1 | 1 |
| Variant 2 | 2 | 2 |
| Variant 3 | 2 | 1 |
| Variant 4 | 2 | 2 |
| Variant 5 | — | 3 |

All publications and patent applications mentioned in the specification are indicative of the level of skill of those skilled in the art to which the embodiments pertain. All publications and patent applications are herein incorporated by reference to the same extent as if each individual publication or patent application was specifically and individually indicated to be incorporated by reference.

Although the foregoing embodiments have been described in some detail by way of illustration and example for purposes of clarity of understanding, it will be obvious that certain changes and modifications may be practiced within the scope of the appended claims.

That which is claimed:

1. A method of determining whether a gene is active against a target pest, the method comprising the steps of:
a. creating a BBMV from the brush border membrane of a target pest wherein BBMV contains a detectable within its lumen;
b. bringing into contact a candidate gene with the BBMV of step (a);
c. evaluating whether or not the candidate gene causes leakage of the BBMV; and
d. thereby, determining whether a gene is active against a target pest.

2. A BBMV comprising a protein receptor from a target pest gut.

3. The BBMV of claim 2, wherein the protein receptor is from a target pest comprising Lepidoptera, Diptera, Hemiptera, and Coleoptera and Nematoda.

4. The method of claim 1, wherein the BBMV is created from a target pest comprising Lepidoptera, Diptera, Hemiptera, and Coleoptera and Nematoda.

5. The BBMV of claim 2, wherein the protein receptor is ABCC2-Cry1Ac or ABCB1-Axmi22z.

6. The BBMV of claim 2, wherein the protein receptor is comprised from gustatory receptors, odorant receptors, ionotropic receptors, transient receptors, potential channel receptors, neuropeptide receptors and serotonin receptors.

7. A method of identifying a pesticidal protein that binds and/or is active against an insect gut receptor, the method comprising the steps of:
a. creating a BBMV from the brush border membrane of a target pest wherein the BBMV contains a detectable within its lumen;
b. the BBMV of (a) further comprising a protein receptor from the gut of an insect;
c. bringing into contact a candidate gene with the BBMV of step (b);
d. evaluating whether or not the candidate gene causes leakage of the BBMV; and
e. thereby, determining whether a pesticidal protein binds/and is active against a insect gut receptor.

8. The method of claim 7, wherein the BBMV is created from a target pest comprising Lepidoptera, Diptera, Hemiptera, and Coleoptera and Nematoda.
